(19) United States
(12) Patent Application Publication
(10) Pub. No.: US 2013/0123185 A1
(43) Pub. Date: May 16, 2013

(54) METHODS OF TREATING AND DIAGNOSING ACUTE CHEST SYNDROME

(71) Applicants: Solomon Ofori-Acquah, Decatur, GA (US); Samit Ghosh, Atlanta, GA (US)

(72) Inventors: Solomon Ofori-Acquah, Decatur, GA (US); Samit Ghosh, Atlanta, GA (US)

(73) Assignee: EMORY UNIVERSITY, Atlanta, GA (US)

(21) Appl. No.: 13/810,281

(22) PCT Filed: Aug. 5, 2011
(86) PCT No.: PCT/US11/46872

§ 371 Date: Jan. 15, 2013

Related U.S. Application Data
(60) Provisional application No. 61/371,188, filed on Aug. 6, 2010.

(51) Int. Cl. A61K 38/17; G01N 33/48; A61K 31/216

(52) U.S. Cl. CPC A61K 38/1709; A61K 31/216; G01N 33/48
USPC 514/15.3; 514/538; 73/61.41

(57) ABSTRACT

In certain embodiments, the disclosure relates to methods of treating or preventing organ inflammation or failure such as pulmonary inflammation comprising administering a heme scavenger such as hemopexin to a subject in need thereof. The subject may be diagnosed with higher than normal levels of protein-free plasma heme or a hemolytic disorder such as sickle cell disease, autoimmune hemolytic anemia, or paroxysmal nocturnal hemoglobinuria or acute lung injury or acute chest syndrome. Typically, the hemopexin is recombinant human hemopexin.

This application claims priority of U.S. provisional application 61/371,188 filed Aug. 6, 2010, hereby incorporated by reference.

### BACKGROUND

Sickle cell disease (SCD) is a genetic blood disorder in which the red blood cells take on a rigid, sickle shape, resulting in various acute and chronic complications that cause morbidity and mortality in those that are afflicted. SCD affects an estimated 100,000 people in the United States (US) and millions more around the world (Weatherall D J. Genomics and global health: time for a reappraisal. Science. 2003; 302(5645):597-599; Weatherall D J. Hemoglobinopathies worldwide: present and future. Curr Mol Med. 2008; 8(7):592-599); Ashley-Koch A, et al. Sickle hemoglobin (HbS) allele and sickle cell disease: a HuGE review. Am J Epidemiol. 2000; 151(9):839-845). The human and economic costs of SCD continues to grow in the US as nearly 2,000 newborns are identified with this disorder every year, more than the combined number for all other genetic disorders. There is currently no widely available cure for SCD, which continues to exert a global burden on society, particularly in sub-Saharan Africa (Weatherall D J. Genomics and global health: time for a reappraisal. Science. 2003; 302(5645):597-599; Weatherall D J. Hemoglobinopathies worldwide: present and future. Curr Mol Med. 2008; 8(7):592-599).

SCD is associated with various complications. The disease can result in severe hemolysis, in which red blood cells break down at an increased rate, releasing free heme into the bloodstream. Additionally, SCD can cause vaso-occlusion, in which sickled red blood cells obstruct capillaries and restrict blood flow, resulting in pain and ischemia. This can lead to infarction in various tissues and organs.

However, the leading cause of death in patients with SCD is acute chest syndrome (ACS) (Platt O S, et al. Mortality in sickle cell disease. Life expectancy and risk factors for early death. N Engl J Med. 1994; 330(23): 1639-1644; Vichinsky E P. Comprehensive care in sickle cell disease: its impact on morbidity and mortality. Semin Hematol. 1991; 28(3):220-226; Vichinsky, et al. The National Acute Chest Syndrome Study G. Causes and Outcomes of the Acute Chest Syndrome in Sickle Cell Disease. N Engl J Med. 2000; 342(25):1855-1865). ACS is a devastating complication in SCD patients in which inflammation of the lungs is characterized by pulmonary infiltrate, alveolar consolidation, and occlusions in pulmonary microcirculation. This lung inflammation results in hypoxemia, respiratory distress, and often death (Platt O S, et al. Mortality in sickle cell disease. Life expectancy and risk factors for early death. N Engl J Med. 1994; 330(23):1639-1644; Castro O, et al. The acute chest syndrome in sickle cell disease: incidence and risk factors. The Cooperative Study of Sickle Cell Disease. Blood. 1994; 84(2):643-649; Vichinsky E P, et al. Causes and outcomes of the acute chest syndrome in sickle cell disease. National Acute Chest Syndrome Study Group. N Engl J Med. 2000; 342(25):1855-1865). ACS occurs in at least one third of all patients with SCD, and continues to have a high mortality rate among those that are affected (Castro O, et al. The acute chest syndrome in sickle cell disease: incidence and risk factors. The Cooperative Study of Sickle Cell Disease. Blood. 1994; 84(2):643-649).

Currently, the disease process of ACS is poorly understood in the field. ACS is thought to be initially triggered by other complications, such as vaso-occlusive pain crises, infection, or fat emboli; however, patients often continue to deteriorate rapidly even when the initial complication has been treated. Furthermore, in the majority of patients, none of these associated factors are identified (Vichinsky E P, et al. The National Acute Chest Syndrome Study G. Causes and Outcomes of the Acute Chest Syndrome in Sickle Cell Disease. N Engl J Med. 2000; 342(25):1855-1865). Additionally, although ACS has historically been considered a vaso-occlusive disease (Yater W, Hansmann G. Sickle cell anemia: a new cause of cor pulmonale: report of two cases with numerous disseminated occlusions of the small pulmonary arteries. Am J Med Sci. 1936; 191:474-484; Kato G J, et al. Deconstructing sickle cell disease: reappraisal of the role of hemolysis in the development of clinical subphenotypes. Blood Rev. 2007; 21(1):37-47); Steinberg M H. Sickle cell anemia, the first molecular disease: overview of molecular etiology, pathophysiology, and therapeutic approaches. Scientific WorldJournal. 2008; 8:1295-1324), there is a well-recognized significant drop in the concentration of hemoglobin (Hb) at the time of diagnosis (Vichinsky E P, et al. N Engl J Med. 2000; 342(25):1855-1865; Davies S C, Luce P J, Win A A, Riordan J F, Brozovic M. Acute chest syndrome in sickle-cell disease. Lancet. 1984; 1(8367):36-38; Sprinkle R H, et al. Acute chest syndrome in children with sickle cell disease. A retrospective analysis of 100 hospitalized cases. Am J Pediatr Hematol Oncol. 1986; 8(2):105-110; Koren et al. Acute chest syndrome in children with sickle cell anemia. Pediatr Hematol Oncol. 1990; 7(1):99-107; van Agtmael et al. Acute chest syndrome in adult Afro-Caribbean patients with sickle cell disease. Analysis of 81 episodes among 53 patients. Arch Intern Med. 1994; 154(5):557-561; Gladwin M T, et al. The acute chest syndrome in sickle cell disease. Possible role of nitric oxide in its pathophysiology and treatment. Am J Respir Crit Care Med. 1999; 159(5 Pt 1):1368-1376). This acute anemia is likely due to hemolysis, which can be caused by several of the factors associated with ACS, such as infection (Rother et al. The clinical sequelae of intravascular hemolysis and extracellular plasma hemoglobin: a novel mechanism of human disease. JAMA. 2005; 293(13):1653-1662). To date, no study has demonstrated a causal role for any of the factors traditionally associated with ACS.

Mechanical ventilation is necessary to support patients with severe ACS; however, this process causes inevitable damage to the lung. In addition to respiratory support for patients, a standardized treatment protocol for acute chest syndrome will also include antibiotic therapy, bronchodilator therapy, fluid and pain management, and blood transfustions (Vichinsky E P, et al. Causes and outcomes of the acute chest syndrome in sickle cell disease. National Acute Chest Syndrome Study Group. N Engl J Med. 2000; 342(25):1855-1865). Other possible treatment options for ACS, often by reducing incidence through treating sickle cell disease itself, have been contemplated.

U.S. Pat. No. 5,626,884 provides for a specific vitamin regiment to increase normal red blood cell production in order to treat sickle cell disease. U.S. Pat. No. 7,026,344 provides for the use of 5-lipoxygenase inhibitor to treat sickle cell disease. U.S. Pat. No. 6,982,154 provides for the use of modified annexin proteins to treat sickle cell disease. U.S. Pat. No. 6,355,661 provides for the use of protected organic aldehyde to treat sickle cell disease. U.S. Pat. No. 7,538,193 provides for the use of nitric oxide-modified hemoglobins to treat sickle cell disease. U.S. Pat. No. 7,329,543 provides for the use of red blood cells loaded with S-nitrosothiol to treat sickle cell disease. U.S. Pat. No. 6,515,001 provides for the use of interleukin-8 receptor ligand drugs to treat inflammatory and autoimmune diseases. PCT Publication WO 2009/140383 provides for the use of aptamers that bind to P-selectin as coagulation, thrombotic, inflammatory, and metastic disease therapeutics. However, because no certain causal role has been found for any factor of ACS, an optimum therapy has remained elusive, and ACS continues to pose a major global health concern. Thus, there is a need in the field for determining and targeting the causal factors of ACS to develop novel strategies to effectively prevent, treat, and diagnose ACS.

### SUMMARY

In certain embodiments, the disclosure relates to methods of treating or preventing organ inflammation or failure, such as pulmonary inflammation, comprising administering an effective amount of a heme scavenger to a subject in need thereof. The heme scavenger can be a hemopexin or hemopexin family protein or glycoprotein, or mutants, derivatives, variants, hybrids, homologs, substituted forms, chimeras, fusions, or forms with substantial identity to a hemopexin. The subject may be diagnosed with, at risk of, higher than normal levels of protein-free plasma heme or a hemolytic disorder such as sickle cell disease, autoimmune hemolytic anemia, malaria, or paroxysmal nocturnal hemoglobinuria or acute lung injury or acute respiratory distress syndrome or acute chest syndrome or exhibiting symptoms thereof.

The hemopexin or hemopexin family proteins/glycoproteins may be naturally isolated recombinant human, non-human species, non-mammalian species, or chimeric. Typically the subject is a human. The hemopexin or hemopexin family protein or glycoprotein can be of any species, but in certain embodiments is human hemopexin (e.g., see GenBank: AAA58678.1). The hemopexin or hemopexin family protein or glycoprotein can also be a precursors, a portion, or an isoform of a hemopexin such as a human hemopexin. Other contemplated hemopexin or hemopexin family proteins or glycoproteins include matrix metalloproteinases: MMP1, MMP2, MMP3, MMP8, MMP9, MMP10, MMP11, MMP12, MMP13, MMP14, MMP15, MMP16, MMP17, MMP19, MMP20, MMP21, MMP24, MMP25, MMP27, MMP28, PRG4 (proteoglycan 4), and VTN (vitronectin).

In certain embodiments, the disclosure relates to methods of treating or preventing pulmonary inflammation comprising administering a TLR4 antagonist to a subject in need thereof. The subject may be diagnosed with higher than normal levels of protein-free plasma heme or a hemolytic disorder such as sickle cell disease, autoimmune hemolytic anemia, or malaria, or paroxysmal nocturnal hemoglobinuria or acute lung injury or acute respiratory distress syndrome or acute chest syndrome. Examples of contemplated TLR4 antagonist include eritoran and resatorvid. Other contemplated compounds include naloxone, naltrexone, (+)-naloxone, (+)-naltrexone, underacylated lipid A structures (containing four or five fatty acids), LPS from the photosynthetic bacterium Rhodobacter sphaeroides (LPS-RS), ibudilast, propentofylline, and amitriptyline.

In certain embodiments, the disclosure relates to methods of identifying a heightened risk of developing pulmonary inflammation comprising measuring plasma hemopexin in a subject and correlating a lower than normal level of hemopexin to a heightened likelihood of developing pulmonary inflammation. Typically the method further comprises the step of reporting the plasma hemopexin level or a heightened risk of developing acute pulmonary inflammation to the subject or a medical professional.

The present disclosure provides for methods of preventing and treating acute pulmonary inflammation, and specifically acute chest syndrome (ACS), in a subject. Generally, these methods target the activation of toll-like receptor 4 (TLR-4) in a subject, and specifically the activation of TLR-4 by free heme in the bloodstream. Thus, methods are provided including administering an agent that reduces free heme in the bloodstream to a subject in need thereof. These methods can reduce or avoid symptoms of acute pulmonary inflammation and more specifically of reducing or avoiding symptoms of ACS. More specifically, the present disclosure provides for the use of free heme-scavenging compounds, such as heme oxygenase-1 (HO-1), hemopexin, albumin, haptoglobin, and other plasma proteins, to prevent and/or treat ACS.

The present disclosure also provides for methods of preventing and treating acute ACS that specifically target toll-like receptor 4 (TLR-4) to reduce or avoid symptoms of ACS in a subject. More specifically, the present disclosure provides for the use of compounds that are TLR-4 antagonists, such as eritoran, resatorvid, and TLR-4 monoclonal antibody to prevent and treat ACS. The present disclosure also provides for methods of preventing and treating ACS that target nuclear factor kappa-light-chain-enhancer of activated B cells (NF-κB), which is activated by free heme-activated TLR-4 to produce the acute inflammatory condition in ACS. More specifically, the present disclosure provides for the use of compounds that inhibit the activation of NF-κB, such as curcumin, and analogues thereof, to prevent and treat ACS.

In certain embodiments, compositions comprising such agents are provided to a subject at risk of an acute pulmonary inflammation. Some of such subjects are carriers of at least one sickle cell mutation. Some of such subjects have been diagnosed with sickle cell disease. Some of such subjects have been infected with at least one virus or bacteria. In some embodiments, such a subject has not been diagnosed with sickle cell disease. In certain embodiments, the subject has suffered an acute lung injury. In some embodiments, such an injury is septic, or non-septic.

The present disclosure also provides for methods of diagnosing susceptibility to and/or severity of ACS in a patient. Specifically, the present disclosure provides the measuring of plasma levels of HO-1 and/or hemopexin, which are contemplated by the present disclosure to be altered in response to free heme, for predicting susceptibility to and the severity of ACS.

### BRIEF DESCRIPTION OF THE DRAWINGS

FIG. 1a is a graph showing plasma heme (i.e. free heme) levels (μM) in Townes sickle cell trait mice (AS, open square) and Townes sickle cell disease mice (SS, closed square) that have (+) or have not (−) been intravenously injected with extracellular hemin (70 μmoles/kg).

FIG. 1b is a graph showing hematocrit levels (%) in AS (open square) and SS mice (closed square) that have (+) or have not (−) been injected with hemin (70 μmoles/kg).

FIG. 1 c is a graph showing survival rates (%) of Townes AS (open circle, n=9) and SS (closed circle, n=8) mice and survival duration (min) following hemin injection (70 μmoles/kg).

FIG. 1 d is a graph showing survival rates (%) and survival duration (min) of Berkeley sickle cell disease mice (SS) injected with saline (open square, n=6), Berkeley sickle cell disease mice injected with hemin at 70 μmoles/kg (closed circle, n=6), and Berkeley mice hemizygous for the sickle cell gene injected with hemin at 70 μmoles/kg (open circle, n=6).

FIG. 2a is a photograph of the lungs of an SS mouse that succumbed to hemin injection at 70 μmoles/kg.

FIG. 2b is a photograph of the lungs of an AS mouse that survived hemin injection at 70 μmoles/kg.

FIGS. 3a and 3b are photomicrographs of lung sections of a SS mouse that succumbed to hemin injection at 70 μmoles/kg.

FIGS. 3c and 3d are photomicrographs of lung sections of an AS mouse that survived hemin injection at 70 μmoles/kg.

FIG. 4a depicts a mouse wearing a non-invasive neck collar clip sensor to detect signals from the carotid arteries in order to correlate oxygen saturation levels with lung pathology.

FIG. 4b is a graph showing real-time measurements of oxygen saturation (SpO₂%, open circle) and breathing rate (breaths per minute, closed circle) in SS mice before (0-60 min) and after (60-120 min) intravenous injection of hemin at 70 μmoles/kg.

FIG. 4c is a graph showing real-time measurements of oxygen saturation (SpO₂%, open circle) and breathing rate (breaths per minute, closed circle) in AS mice before (0-60 min) and after (60-120 min) intravenous injection of hemin at 70 μmoles/kg.

FIG. 4d is a graph showing the combined data of oxygen saturation (SpO₂%) over time (min) in SS mice before (open circle) and after (closed circle) intravenous injection of hemin at 70 μmoles/kg.

FIG. 4e is a graph showing the combined data of oxygen saturation (SpO₂%) over time (min) in AS mice before (open circle) and after (closed circle) intravenous injection of hemin at 70 μmoles/kg.

FIG. 5a is a graph showing the level of vascular leakage (OD at 620 nm) in the lungs of AS (open circle) and SS (closed circle) mice of various ages (weeks).

FIG. 5b is a graph showing survival rates (%) and survival duration (min) of young (5-7 weeks) SS mice (open circle, n=15) and adult (13-27 weeks) SS mice (closed circle, n=14) following intravenous injection of hemin at 70 μmoles/kg.

FIG. 5c is a graph showing plasma heme oxygenase-1 (HO-1) concentrations (ng/ml) over activity levels (nm bilirubin/mg/hr) in SS (closed circle) and AS (open circle) mice. FIG. 5d is a graph showing plasma HO-1 concentrations (ng/ml) in young (5-7 weeks), adult (13-27 weeks), and middle-aged (44-54 weeks) SS mice.

FIG. 5e is a graph showing plasma HO-1 concentrations (ng/ml) in sickle cell disease patients (SS, closed circle) and patients that do not have sickle cell disease (AA, closed square).

FIG. 6a is a graph showing plasma heme (i.e. free heme) concentrations (mM) in wild type (C57BL/6J, open square) and TLR-4 mutant (B6.B10ScN-Tlr4^{LPS-del}/JthJ, closed square) mice that have (+) or have not (−) been administered an intravenous injection of a normally lethal dose of hemin (210 μmoles/kg).

FIG. 6b (i) is a photograph of a lung from a TLR4 mutant mouse that survived injection with a normally lethal dose of hemin (210 μmoles/kg).

FIG. 6b(ii) is a photograph of the lung of a normal mouse expressing TLR4, which succumbed to 210 μmoles/kg of hemin.

FIG. 6c (i) is a photomicrograph of the lung of a TLR4 mutant mouse that survived 210 μmoles/kg of hemin.

FIG. 6c (ii) is a photomicrograph of the lung of a normal mouse that succumbed to 210 μmoles/kg of hemin.

FIG. 6d is a graph showing the survival rates (%) and survival duration (min) of wild type (C57BL/6J, closed circle, n=11) and TLR-4 mutant (B6.B10ScN-Tlr4^{LPS-del}/JthJ, open circle, n=9; C3H/HeJ, open square, n=5) mice following intravenous injection of a normally lethal dose of hemin (210 μmoles/kg).

FIG. 7a is a western blot showing expression of TLR-4 and Actin in the absence (0 μM) or presence (10 μM) of hemin in pulmonary microvascular endothelial cells (PMVECs).

FIG. 7b is an immunoflorescence staining of PMVECs showing the location of p65 NF-κB in the absence (0 μM, left) or presence (10 μM, right) of hemin.

FIG. 7c is a western blot showing the expression of p65 NF-κB, Laminin B, and LDH in PMVECs exposed to 0 μM, 5 μM, or 10 μM hemin.

FIG. 7d is a graph showing NF-κB activity (fold) via a luciferase reporter gene driven by a promoter harboring NF-κB cis elements in PMVECs treated with vehicle (0 μM hemin), 5 μM or 10 μM hemin, or tumor necrosis factor alpha (TNF-α) at 20 ng/ml.

FIG. 7e is a graph showing TNF-α production (μg/ml) in PMVECs treated with 0 μM or 10 μM hemin.

FIG. 8 shows data suggesting heme induces a lethal form of ALI in SS mice. (a) Steady-state concentration of total plasma heme in transgenic AA (n=8), AS (n=20) and SS (n=21) mice. (b) Steady-state concentration of protein-free plasma heme in AA (n=8), AS (n=21) and SS (n=21). (c) Total and protein-free plasma heme in SS mice after intravenous administration of heme (0-70 μmoles/kg) (n=6-15). (d) Concentration of total plasma heme and protein-free plasma heme in AA, AS & SS mice intravenously administered heme (70 μmoles/kg, n=6-15). (e, f) Percentage oxygen saturation and breath rate in AS and SS mice (n=6) before and after injection of heme. Each mouse was monitored awake using a pulse oximeter for 1 h before and after heme injection (70 μmoles/kg). Each data point is the mean SpO2 or breath rate for 300 recordings made in the 5-minute intervals indicated. Number of animals survived at each time point is indicated. (g) Survival of SS mice treated with heme (17.5-70 μmoles/kg, n=6-13). (h) Survival of AA (n=6) and AS (n=16) mice treated with 70 μmoles/kg of heme.

FIG. 9 show a representative postmortem H&E staining of lung tissues of saline and heme treated SS mice. (Top) Substantial vascular congestion, alveolar edema, alveolar wall thickening and hemorrhage are indicated by arrows. Scale bar, alveolar wall thickening; 50 μm, all other images; 100 μm. (Bottom) Semi-quantitative analysis of histological changes based on

scoring of H&E stained sections in SS mice treated with saline (n=6) or heme (n=12). *p<0.05, **p<0.01 and ***p<0.001.

FIG. 10 shows data suggesting plasma hemopexin level in SCD is a risk factor for sudden lethality (a) Plasma albumin level and albumin/total protein ratio in AA, AS and SS (n=7-8). (b) Plasma hemopexin level in AA, AS and SS mice (n=7-8). (c) Acute elevation of circulating heme rapidly depletes the hemopexin stores in SS mice. Plasma samples were analyzed at baseline (0) and 5, 15 and 30 min after heme injection (35 moles/kg, n=6 at each time point). (d) Distribution of plasma hemopexin level in SCD mice combined from two different genetic models (Townes, n=38, Berkeley, n=40). Mice were arbitrarily divided into three groups based on hemopexin level. (e) Survival of SS mice injected with heme (35 moles/kg) with different baseline plasma hemopexin level. (f) Expected mortality probability as in (e), plotted as a function of baseline plasma hemopexin level.

FIG. 11 shows data suggesting acute prophylaxis with recombinant human hemopexin (rhHx) protects SS mice from heme induced lethality. Mice were injected with rhHx (n=6), vehicle (n=5) or human IgG (n=3) immediately before administration of heme (35 μmoles/kg). *p<0.05, **p<0.01 and ***p<0.001.

FIG. 12 shows data suggesting heme induced lethal ALI requires TLR4. (a) Concentration of total plasma heme and protein free plasma heme in C57BL/6J (n=6), B6.B10ScN-Tlr4lps-del/JthJ (n=5) and C3H/HeJ (n=8) mice at steady-state in the absence of heme (−), and 5 min after intravenous administration of heme (+); 210 μmoles/kg. (b, c) Oxygen saturation and breath rate in C57BL/6J and C3H/HeJ mice (n=6) monitored non-invasively, as in 8e & f, before and after injection of heme (210 μmoles/kg). Number of animals at various times during the experiment is indicated. (d) Survival of C57BL/6J (n=17), B6.B10ScNTlr4lps-del/JthJ (n=9) and C3H/HeJ (n=16) after heme administration.

FIG. 13 shows H&E staining of lung tissues of C57BL/6J, B6.B100ScN-Tlr4lps-del/JthJ and C3H/HeJ mice. (Top) Tissues were collected immediately after death in C57BL/6J, and in TLR4 mutants that were sacrificed 2 h after heme administration. Lung injury score in saline (n=3) and heme treated C57BL/6J (n=6), B6.B100ScN-Tlr4lps-del/JthJ (n=5) and C3H/HeJ (n=6) mice (bottom). Each bar represents the mean cumulative histological score based on severity of four different lung injury indicators (edema, hemorrhage, alveolar wall thickening and vascular congestion, scored 0-4). *p<0.05, **p<0.01 and ***p<0.001.

FIG. 14 shows data suggesting esatorvid prevents the development of heme induced ALI in SCD mice. (a, b) The concentration of total and protein-free plasma heme in SS mice following, injections of TAK-242 or intralipid in the absence of exogenous heme. (c, d) Percentage oxygen saturation and breath rate monitored in real-time in SS mice at different stages of the experiment as indicated. Data shown are for a total of nine SS mice (intralipid; n=4, TAK-242; n=5). (e) Survival of SS mice pre-treated with TAK-242 (3 or 5 mg/kg) for 1 h prior to intravenous administration of heme (n=7-13). Control mice were treated with intralipid (n=17). (f) Representative lung H&E staining of SS mice treated with intralipid or TAK-242 one hour prior to administration of heme (35 μmoles/kg). Cumulative lung injury score (n=3). *p<0.05, **p<0.01 and ***p<0.001.

### DETAILED DESCRIPTION

### I. Definitions

As used herein, the term “free heme” refers to extracellular heme and/or hemin found in the blood plasma in circulation within a body.

As used herein, the term “compound” refers to the compositions contemplated by the methods of the present disclosure to be effective in the treatment and/or prophylaxis of acute chest syndrome and/or acute respiratory distress syndrome when administered in an effective amount. Specifically, the term refers to free heme-scavenging and degrading compounds, TLR-4 antagonists, NF-κB antagonists, and any combinations thereof.

As used herein, the term “free heme-scavenging compound” refers to any compound able to bind, deactivate, degrade, and/or otherwise inhibit free heme from producing or helping produce an inflammatory condition within a body.

As used herein, the term “TLR-4 antagonist” refers to any compound able to bind and/or deactivate or otherwise inhibit toll-like receptor 4 from producing or helping produce an inflammatory condition within a body.

As used herein, the term “NF-κB antagonist” refers to any compound able to bind and/or deactivate or otherwise inhibit NF-κB from producing or helping produce an inflammatory condition within a body.

As used herein, “subject” refers to any animal, preferably a human patient, livestock, or domestic pet.

The terms “protein” and “polypeptide” refer to compounds comprising amino acids joined via peptide bonds and are used interchangeably. As used herein, the terms “prevent” and “preventing” include the prevention of the recurrence, spread or onset. It is not intended that the present disclosure be limited to complete prevention. In some embodiments, the onset is delayed, or the severity is reduced.

As used herein, the terms “treat” and “treating” are not limited to the case where the subject (e.g. patient) is cured and the disease is eradicated. Rather, embodiments of the present disclosure also contemplate treatment that merely reduces symptoms, and/or delays disease progression.

As used herein, where “amino acid sequence” is recited herein to refer to an amino acid sequence of a protein molecule. An “amino acid sequence” can be deduced from the nucleic acid sequence encoding the protein. However, terms such as “polypeptide” or “protein” are not meant to limit the amino acid sequence to the deduced amino acid sequence, but include post-translational modifications of the deduced amino acid sequences, such as amino acid deletions, additions, and modifications such as glycolsylations and addition of lipid moieties. The term “portion” when used in reference to a protein (as in “a portion of a given protein”) refers to fragments of that protein. The fragments may range in size from four amino acid residues to the entire amino sequence minus one amino acid.

The term “chimera” when used in reference to a polypeptide refers to the expression product of two or more coding sequences obtained from different genes, that have been cloned together and that, after translation, act as a single polypeptide sequence. Chimeric polypeptides are also referred to as “hybrid” polypeptides. The coding sequences include those obtained from the same or from different species of organisms.

The term “fusion” when used in reference to a polypeptide refers to a chimeric protein containing a protein of interest joined to an exogenous protein fragment (the fusion partner). The fusion partner may serve various functions, including enhancement of solubility of the polypeptide of interest, conferring binding to the haptoglobin receptor, other heme and hemoglobin scavenging receptors and transporters, as well as providing an “affinity tag” to allow purification of the recombinant fusion polypeptide from a host cell or from a supernatant or from both. If desired, the fusion partner may be removed from the protein of interest after or during purification.

The term “homolog” or “homologous” when used in reference to a polypeptide refers to a high degree of sequence identity between two polypeptides, or to a high degree of similarity between the three-dimensional structure or to a high degree of similarity between the active site and the mechanism of action. In a preferred embodiment, a homolog has a greater than 60% sequence identity, and more preferably greater than 75% sequence identity, and still more preferably greater than 90% sequence identity, with a reference sequence. As applied to polypeptides, the term “substantial identity” means that two peptide sequences, when optimally aligned, such as by the programs GAP or BESTFIT using default gap weights, share at least 80 percent sequence identity, preferably at least 90 percent sequence identity, more preferably at least 95 percent sequence identity or more (e.g., 99 percent sequence identity). Preferably, residue positions which are not identical differ by conservative amino acid substitutions.

The terms “variant” and “mutant” when used in reference to a polypeptide refer to an amino acid sequence that differs by one or more amino acids from another, usually related polypeptide. The variant may have “conservative” changes, wherein a substituted amino acid has similar structural or chemical properties. One type of conservative amino acid substitutions refers to the interchangeability of residues having similar side chains. For example, a group of amino acids having aliphatic side chains is glycine, alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Preferred conservative amino acids substitution groups are: valine-leucine-isoleucine, phenylalanine-tyrosine, lysine-arginine, alanine-valine, and asparagine-glutamine. More rarely, a variant may have “non-conservative” changes (e.g., replacement of a glycine with a tryptophan). Similar minor variations may also include amino acid deletions or insertions (in other words, additions), or both. Guidance in determining which and how many amino acid residues may be substituted, inserted or deleted without abolishing biological activity may be found using computer programs well known in the art, for example, DNAStar software. Variants can be tested in functional assays. Preferred variants have less than 10%, and preferably less than 5%, and still more preferably less than 2% changes (whether substitutions, deletions, and so on).

The term “recombinant” when made in reference to a nucleic acid molecule refers to a nucleic acid molecule which is comprised of segments of nucleic acid joined together by means of molecular biological techniques. The term “recombinant” when made in reference to a protein or a polypeptide refers to a protein molecule which is expressed using a recombinant nucleic acid molecule.

### II. Impaired Heme Scavenging Promotes the Development of a Lethal Form of Acute Lung Injury in Sickle Cell Disease

Heme released into the circulation by tissue damage is scavenged by multiple plasma proteins and degraded in the liver. However, excessive heme accumulation causes terminal damage to the liver and other systemic organs. By using humanized mouse models, heme scavenging is shown to severely impaired in sickle cell disease (SCD), resulting in the development of a lethal acute lung injury (ALI). The damage to the lung is caused by unscavenged heme and can be blocked by restoring heme scavenging capacity in mice. Heme induced ALI involves toll-like receptor 4 (TLR4). Consequently, prophylaxis using resatorvid, a TLR4 inhibitor, protected SCD mice from developing ALI despite failing to scavenge heme.

It has been discovered that heme triggers a lethal form of ALI. The steady-state plasma hemopexin level in SCD is a risk factor for heme induced ALI, in a process involving TLR4 signaling. An experimental system mimicking acute hemolysis was developed in SCD that increases the concentration of heme in the bloodstream, and this caused a failure to scavenge heme, and triggered a lethal form of ALI. SCD patients have low plasma hemopexin levels. It has been discovered that transgenic SCD mice have a similar phenotype. Baseline hemopexin levels correlated with survival after heme administration, and recombinant human hemopexin protected SS mice from inevitable lethality. Thus, certain embodiments of the disclosure contemplate diagnostic assays to measure plasma hemopexin in SCD patients, particularly, during the acute phase of the disease may help identify those at a higher risk of developing ACS. A major deficit in plasma albumin level in SS mice was not found. Nonetheless, human albumin is widely used clinically, so it will be interesting to investigate whether 20% human albumin, by binding protein-free heme, will be effective in slowing progression of ACS, while efforts are directed to developing strategies to produce hemopexin for clinical use.

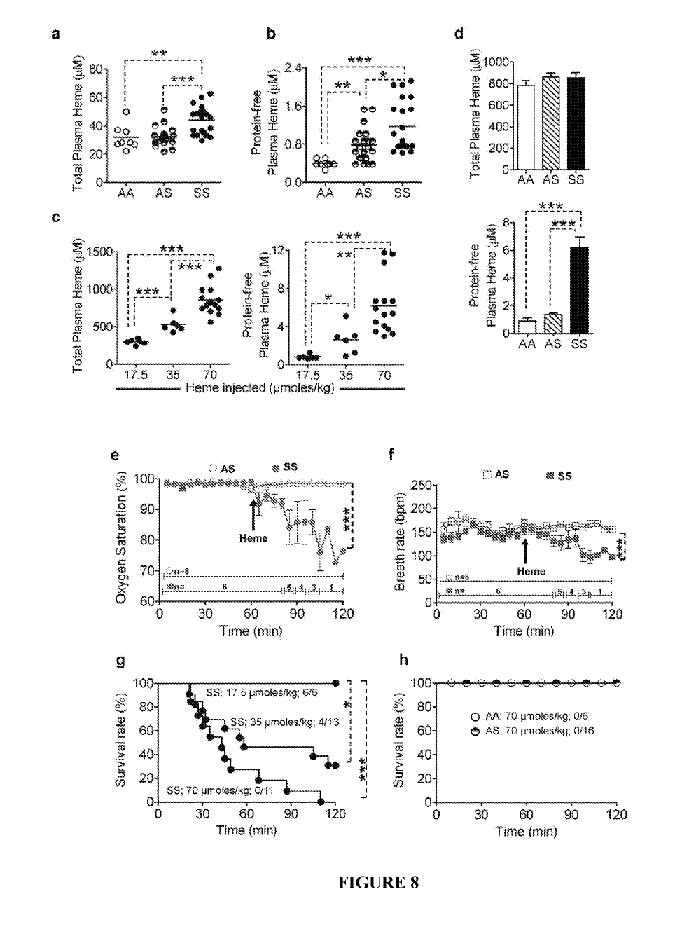

Although heme is known to cause organ damage in multiple systems (liver, heart and kidneys), it has not previously been shown to cause ALI. Others focused primarily on the sequelae due to increased plasma heme have shown that excess heme accumulation causes organ damage in multiple systems. There was no report of lung damage in the study by Larsen et al., Sci Transl Med, 2010, 2(51):51ra71, in which heme was shown to contribute to organ failure in multiple systems. Both the lung and liver were examined, and no liver involvement in the sequelae due to PFPH was found (FIG. 8).

Although it is not intended that certain embodiments of the disclosure are to be limited by any particular mechanism, it is believed that two possible mechanisms of heme induced pathology are at work, a classical pathway involving scavenged heme deposited into tissues that Larsen et al, and others described and an alternative pathway triggered by unscavenged heme, which is identified here for the first time. These two pathways may overlap in disorders such as SCD, in which the amount of heme released into the circulation may be determined by the severity of independent hemolytic events. In the current disclosure, severe/lethal forms of lung injury in were evaluated SCD. It is worth noting that the amount of heme administered to SS mice were not in themselves high, in absolute terms, since the same doses were effectively cleared by AS littermates, without any adverse effects several weeks after the experiments. Using these relatively low doses, an intrinsic problem in SCD was unraveled, namely impaired heme scavenging. This suggests the dire consequences of impaired heme scavenging on lung function. Evidence of a receptor mediated heme induced pathology was also identified.

There are currently no proven interventions to prevent the progression of ACS, and it remains the leading cause of death in SCD. Hydroxyurea and chronic blood transfusion therapies can reduce the incidence of ACS in individuals with a history of this complication, the efficacy of exchange red blood cell transfusion to reduce the severity of ongoing ACS remains unclear. The link between hemolysis and ACS was exploited, particularly in adults who have a more severe phenotype, to create an animal model that recapitulates some of the features of the human disease, notably, pulmonary infiltration, progressive oxygen desaturation, respiratory distress and lethality. Heme, hemopexin and TLR4 have been identified as targets for managing ACS. By targeting the heme/hemopexin and TLR4 axis, progression of ACS may be halted with the potential to reduce morbidity and mortality associated with SCD.

### III. Free Heme-Scavenging Compounds

The method of the present disclosure contemplates that free heme, produced as a result of hemolysis, is a causal factor of ACS. This overall notion challenges a historical belief (Yater W, Hansmann G. Sickle cell anemia: a new cause of cor pulmonale: report of two cases with numerous disseminated occlusions of the small pulmonary arteries. Am J Med Sci. 1936; 191:474-484; Steinberg B. Sickle cell anemia. Arch Pathol. 1930; 9:876-897), reinforced recently by a new model of SCD, which postulates that ACS belongs to a sub-phenotype of SCD that is defined predominantly by vaso-occlusion, and not hemolysis (Kato G J, et al. Deconstructing sickle cell disease: reappraisal of the role of hemolysis in the development of clinical subphenotypes. Blood Rev. 2007; 21(1):37-47). However, it has long been recognized that virtually all patients with ACS have hemolysis during the acute phase of the syndrome. Excess hemoglobin from intravascular hemolysis is oxidized to met-hemoglobin, which in turn readily releases hemin (ferric heme) into the circulation. Indeed, at the time of diagnosis of ACS, virtually all patients have a decreasing concentration of hemoglobin (Vichinsky et al. The National Acute Chest Syndrome Study G. Causes and Outcomes of the Acute Chest Syndrome in Sickle Cell Disease. N Engl J Med. 2000; 342(25):1855-1865; Davies S C, Luce P J, Win A A, Riordan J F, Brozovic M. Acute chest syndrome in sickle-cell disease. Lancet. 1984; 1(8367):36-38; Sprinkle R H, et al. Acute chest syndrome in children with sickle cell disease. A retrospective analysis of 100 hospitalized cases. Am J Pediatr Hematol Oncol. 1986; 8(2): 105-110; Koren et al. Acute chest syndrome in children with sickle cell anemia. Pediatr Hematol Oncol. 1990; 7(1):99-107; van Agtmael et al. Acute chest syndrome in adult Afro-Caribbean patients with sickle cell disease. Analysis of 81 episodes among 53 patients. Arch Intern Med. 1994; 154(5):557-561; Gladwin M T, et al. The acute chest syndrome in sickle cell disease. Possible role of nitric oxide in its pathophysiology and treatment. Am J Respir Crit Care Med. 1999; 159(5 Pt 1):1368-1376), and those who succumb rapidly often have lowest hemoglobin levels at presentation (Vichinsky et al. Acute chest syndrome in sickle cell disease: clinical presentation and course. Cooperative Study of Sickle Cell Disease. Blood. 1997; 89(5):1787-1792).

It is contemplated by the present disclosure that an acute hemolysis, due to some stressor, generates an overabundance of free heme that the SCD patient cannot tolerate or clear, resulting in the inflammatory condition of ACS. Indeed, it is shown by the present disclosure that an acute increase in free heme will induce ACS and mortality in a murine model where mice are homozygous for the human sickle cell disease gene and exhibit the sickle cell disease phenotype (see Example 1).

In certain embodiments of the present disclosure, free heme-scavenging compounds are used in the treatment of acute chest syndrome. It is contemplated by the present disclosure that such compounds have an inhibitory effect on free heme, preventing it from inducing the inflammatory condition in the lungs to cause ACS in sickle cell disease patients. Additionally, in other embodiments, free heme-scavenging compounds are used in the treatment of acute respiratory distress syndrome (ARDS) in patients not afflicted by sickle cell disease, as ARDS is characterized by the same initial stressors, inflammatory progression in the lungs, and symptoms as ACS, the difference being that ARDS occurs in non-sickle cell disease patients. In yet other embodiments, free heme-scavenging compounds are used in the prophylaxis of ACS and/or ARDS.

In certain embodiments of the present disclosure, one of the free heme-scavenging compounds used in treatment and/or prophylaxis is heme-oxygenase-1 (HO-1). HO-1 is an inducible stress protein that degrades free heme, and has anti-inflammatory effects. Indeed, it is shown by the present disclosure that HO-1 plasma concentrations are elevated in SS mice as well as SCD patients compared to AS mice and non-sickle cell disease patients, respectively (see Example 2). However, HO-1 levels in SS mice decrease with age; concurrently, survival of SS mice following intravenous injection of free hemin also decreases with age. Furthermore, HO-1 is not elevated in the lungs of adult SS mice (Ghosh et al. Antioxidant Defense to Hemolysis Is Organ-Specific and Reflects a Heterogeneity in Vascular Permeability in Sickle Cell Disease. Blood. 2009; 114 (suppl 1):1537), and vascular leakage in the lung endothelium is greater in adult SS mice when compared to AS mice and younger SS mice. The above findings (see Example 2) indicate that HO-1 is an important compound which, when absent, allows free heme-induced ACS (and ARDS) to develop. In other embodiments of the present disclosure, one of the free heme-scavenging compounds used in treatment and/or prophylaxis is hemopexin. Hemopexin is a protein that binds free heme for transport to the liver for breakdown, both for iron recovery and to combat oxidative stress. In yet other embodiments of the present disclosure, one of the free heme-scavenging compounds used in treatment and/or prophylaxis is albumin. Albumin is a human serum protein that will also bind free heme for transport to the liver to combat oxidative stress when available hemopexin becomes saturated with bound heme/hemin. In yet other embodiments, one of the free heme-scavenging compounds used in treatment and/or prophylaxis is haptoglobin, a protein also capable of binding free hemoglobin, the precursor of free heme. In some embodiments, the free heme binding compounds are heme-binding DNA aptamers, and other nucleotide-based heme-binding molecule.

In yet other embodiments, the measurement of plasma levels of HO-1 and/or hemopexin is used to diagnose susceptibility of sickle cell disease patient to ACS. In yet other embodiments, the measurement of plasma levels of HO-1 and/or hemopexin is used to diagnose the severity of ACS in sickle cell disease patients. It is also contemplated by the present disclosure that measurement of HO-1 and/or hemopexin plasma levels is used to diagnose susceptibility to and/or severity of ARDS in non-sickle cell disease patients.

### IV. TLR-4 Antagonists and NF-κB Antagonists

The method of the present disclosure contemplates that certain downstream targets are activated by free heme to produce the inflammatory condition of the lung. One of these downstream targets is toll-like receptor-4, which has recently been found to be activated by free heme (Figueiredo et al. Characterization of heme as activator of Toll-like receptor 4. J Biol Chem. 2007; 282(28):20221-20229). Indeed, it is shown by the present disclosure that a mutation in the TLR-4 gene in the murine model allows for 100% survival following an intravenous injection of free hemin at a concentration that is lethal to wild-type mice (see Example 3).

In certain embodiments of the present disclosure, TLR-4 antagonists are used in the treatment of ACS. It is contemplated by the present disclosure that such antagonists will inhibit the free heme-induced protein cascade that results in the lung inflammation of ACS. Additionally, in other embodiments, TLR-4 antagonists are used in the treatment of acute respiratory distress syndrome (ARDS) in patients not afflicted by sickle cell disease, as ARDS is characterized by the same initial stressors, inflammatory progression in the lungs, and symptoms as ACS, the difference being that ARDS occurs in non-sickle cell disease patients. In yet other embodiments, TLR-4 antagonists are used in the prophylaxis of ACS and/or ARDS.

In certain embodiments of the present disclosure, one of the TLR-4 antagonists used in treatment and/or prophylaxis is eritoran. In other embodiments, the compound is any compound described in U.S. Pat. No. 5,750,664. As a TLR-4 antagonist, eritoran has an original indication for the treatment of severe sepsis. In yet other embodiments of the present disclosure, one of the TLR-4 antagonists used in treatment and/or prophylaxis is resatorvid, which also has an original indication for the treatment of severe sepsis. In other embodiments, the compound is one described in U.S. Pat. No. 6,495,604. In still yet other embodiments of the present disclosure, TLR-4 human monoclonal antibody is used to inhibit the action of TLR-4 and thus treat and/or prevent ACS, such as those described in U.S. Pat. No. 7,312,320 and U.S. Pat. No. 7,674,884.

The method of the present disclosure also contemplates that NF-κB is a downstream target of free heme-activated TLR-4 in the signal cascade for lung inflammation in ACS. Indeed, the present disclosure has shown that free heme activates NF-κB in pulmonary endothelial cells (See Example 4).

In certain embodiments of the present disclosure, NF-κB antagonists are used in the treatment of ACS. It is contemplated by the present disclosure that such antagonists will inhibit the free heme-induced protein cascade that results in the lung inflammation of ACS. Additionally, in other embodiments, NF-κB antagonists are used in the treatment of acute respiratory distress syndrome (ARDS) in patients not afflicted by sickle cell disease, as ARDS is characterized by the same initial stressors, inflammatory progression in the lungs, and symptoms as ACS, the difference being that ARDS occurs in non-sickle cell disease patients. In yet other embodiments, NF-κB antagonists are used in the prophylaxis of ACS and/or ARDS. In still yet other embodiments, the NF-κB antagonists used in treatment and/or prophylaxis are curcumin analogs. In still yet other embodiments, the NF-κB antagonist used in treatment and/or prophylaxis is sulfasalazine.

### V. Therapies and Administration

The present disclosure provides a method of treatment, prophylaxis, and diagnosis for ACS as well as ARDS. Humans suffering from ACS or ARDS can be treated by administering to the patient an effective amount of the compounds used in the present disclosure. Administration may be through a pharmaceutically acceptable salt or prodrug thereof in the presence of a pharmaceutically acceptable carrier or diluent, which upon administration to the recipient is capable of providing the compounds used in the present disclosure. The compounds used in the present disclosure can be administered by any appropriate route, for example, orally, intravenously, parenterally, enterally, intradermally, subcutaneously, rectally, topically, and/or intranasally. The compounds of the present disclosure may be administered before, upon, and/or after the onset of the condition to be alleviated, and may be administered on various dosing regiments.

The amount of compound in the drug composition will depend on absorption, distribution, metabolism, and excretion rates of the drug as well as other factors known to those of skill in the art. Dosage values will also vary with the severity of the condition to be alleviated. The compounds may be administered once, or may be divided and administered over intervals of time. It is to be understood that administration may be adjusted according to individual need and profession judgment of a person administrating or supervising the administration of the compounds used in the present disclosure.

The dose of the inventive composition administered to an individual (such as human) will vary with the particular composition, the method of administration, and the particular disease being treated. The dose should be sufficient to effect a desirable response, such as a therapeutic or prophylactic response against a particular disease or condition. For example, the dosage of a compound of the disclosure administered can be about 1 to about 1000 mg/m², about 1 to about 500 mg/m², about 1 to about 300 mg/m², including for example about 10 to about 300 mg/m², about 30 to about 200 mg/m², and about 70 to about 150 mg/m². Typically, the dosage of a compound of the disclosure in the composition can be in the range of about 50 to about 200 mg/m² when given on a 3 week schedule, or about 10 to about 100 mg/m² when given on a weekly schedule. In addition, if given in a metronomic regimen (e.g., daily or a few times per week), the dosage may be in the range of about 1-50 mg/m².

Dosing frequency for the composition includes, but is not limited to, at least about any of once every three weeks, once every two weeks, once a week, twice a week, three times a week, four times a week, five times a week, six times a week, or daily. In some embodiments, the interval between each administration is less than about a week, such as less than about any of 6, 5, 4, 3, 2, or 1 day. In some embodiments, the interval between each administration is constant. For example, the administration can be carried out daily, every two days, every three days, every four days, every five days, or weekly. In some embodiments, the administration can be carried out twice daily, three times daily, or more frequent. Administration can also be continuous and adjusted to maintaining a level of the compound within any desired and specified range.

The administration of the composition can be extended over an extended period of time, such as from about a month up to about three years. For example, the dosing regime can be extended over a period of any of about 2, 3, 4, 5, 6, 7, 8, 9, 10, 11, 12, 18, 24, 30, and 36 months. In some embodiments, there is no break in the dosing schedule. In some embodiments, the interval between each administration is no more than about a week.

The compositions described herein can be administered to an individual (such as human) via various routes, including, for example, intravenous, intra-arterial, intraperitoneal, intrapulmonary, oral, inhalation, intravesicular, intramuscular, intra-tracheal, subcutaneous, intraocular, intrathecal, transmucosal, and transdermal. For example, the inventive composition can be administered by inhalation to treat conditions of the respiratory tract. The composition can be used to treat respiratory conditions such as pulmonary fibrosis, broncheolitis obliterans, lung cancer, bronchoalveolar carcinoma, and the like. In one embodiment of the disclosure, nanoparticles (such as albumin nanoparticles) of the inventive compounds can be administered by any acceptable route including, but not limited to, orally, intramuscularly, transdermally, intravenously, through an inhaler or other air borne delivery systems and the like.

When preparing the composition for injection, particularly for intravenous delivery, the continuous phase preferably comprises an aqueous solution of tonicity modifiers, buffered to a pH range of about 5 to about 8.5. The pH may also be below 7 or below 6. In some embodiments, the pH of the composition is no less than about 6, including for example no less than about any of 6.5, 7, or 8 (such as about 7.5 or 8).

The compounds used in the present disclosure may be administered individually, or in combination with or concurrently with one or more other compounds used in other embodiments of the present disclosure. Additionally, compounds used in the present disclosure may be administered in combination with or concurrently with other therapeutics for ACS and/or ARDS, including, but not limited to, antibiotics, antivirals, immunosuppressants, mechanical therapies (e.g. lung ventilation), and the like. Furthermore, the compounds used in the present disclosure may be administered in combination with or concurrently with therapies for sickle cell disease. Common medicines used to treat pain crises associated with sickle cell disease include acetaminophen, nonsteroidal anti-inflammatory drugs (NSAIDs), and narcotics such as meperidine, morphine, oxycodone, and others. Hydroxyurea is also often used in treatment of sickle cell and the present compositions can be formulated in combination, or administered in combination or alternation with hydroxyurea. In some embodiments, the compounds are administered in combination or alternation with an antibiotic. In certain other embodiments, the compounds are administered in combination or alternation with butyric acid, nitric oxide or decitadine.

### VI. Examples

### Example 1

### Acute Increase in Plasma (Free) Heme Causes ACS in SCD Mice

The murine model was used to determine the relationship between free heme and acute chest syndrome in subjects suffering from sickle cell disease. The Townes model has the mouse α- and β-globin genes replaced with the corresponding human genes (Wu et al. Correction of sickle cell disease by homologous recombination in embryonic stem cells. Blood. 2006; 108(4):1183-1188; Hanna et al. Treatment of sickle cell anemia mouse model with iPS cells generated from autologous skin. Science. 2007; 318(5858):1920-1923). The Townes sickle cell disease mouse (SS) is homozygous for the human β^{S}-globin, while the Townes sickle cell trait mouse (AS) control has one β^{S} and one β^{A}-globin gene. In the Berkeley mouse model, the sickle cell disease mouse (SS) has deletions of the mouse globin genes and carrying a DNA construct containing the human α- and β^{S}-globin genes (Paszty et al. Transgenic knockout mice with exclusively human sickle hemoglobin and sickle cell disease. Science. 1997; 278(5339):876-878). The Berkeley control mouse has only a single human β^{S}-globin gene and is therefore a hemizygote. Disease mice (SS) of both models have features of human SCD, including irreversibly sickle red blood cells, severe anemia, and multi-organ pathology (Hanna et al. Treatment of sickle cell anemia mouse model with iPS cells generated from autologous skin. Science. 2007; 318(5858):1920-1923; Paszty et al. Transgenic knockout mice with exclusively human sickle hemoglobin and sickle cell disease. Science. 1997; 278(5339):876-878).

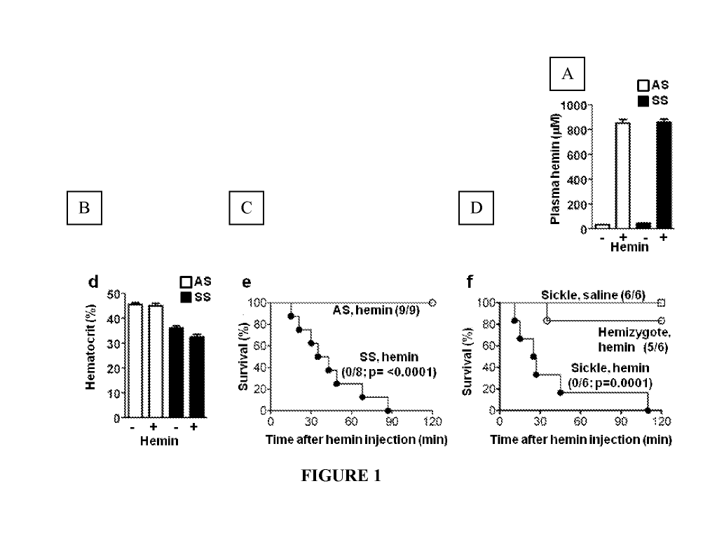

SS and control mice were intravenously injected with hemin using a dose (70 μmoles/kg) shown in a previous study to increase lung permeability by 2-fold, without causing any significant lung pathology or death in non-sickle cell disease mice (Vinchi et al. Hemopexin prevents endothelial damage and liver congestion in a mouse model of heme overload. Am J Pathol. 2008; 173(1):289-299). This dose increased the plasma concentration of heme by 20-fold (FIG. 1a) and did not significantly change the hematocrit in SS or control mice (FIG. 1b), confirming that the hemin injected was extracellular (i.e., free heme). Hemin injection caused sudden death in both the Townes and Berkeley transgenic mouse models of SCD, while control littermates (Townes AS mouse, and Berkeley hemizygote mouse) of both models survived, yielding a highly significant difference in survival that was calculated using the log-rank (Mantel-Cox) test (FIGS. 1 c and 1d). This experiment had a power of 90% to detect a 25% difference in survival with a significance level (alpha) of 0.05 (two-tailed) assuming 10% death in the control group in a total sample size of 20. Power analysis was performed using StatMate 2.0.

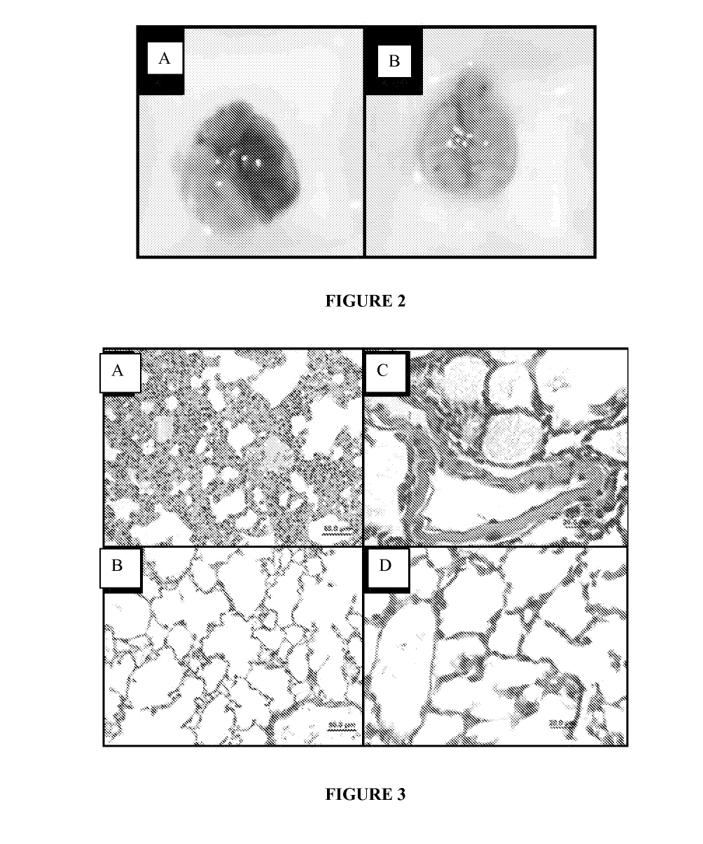

Post mortem analysis implicated the lung in the sudden death caused by free heme. This was evident by gross pulmonary infiltration in SS mice (FIG. 2a), that was never found in control mice sacrificed 5 hours after surviving the hemin injection (FIG. 2b). Histological analysis revealed multiple pathologies consistent with acute respiratory distress, including alveolar flooding and severe microvascular occlusions in the SS mice (FIGS. 3a and 3b), compared to a relatively normal lung in AS mice (FIGS. 3c and 3d). Thus, acute elevation of plasma (i.e. free) heme induces pulmonary infiltration, alveolar flooding, severe microvascular occlusion and mortality in SS mice. These findings closely mirror human ACS.

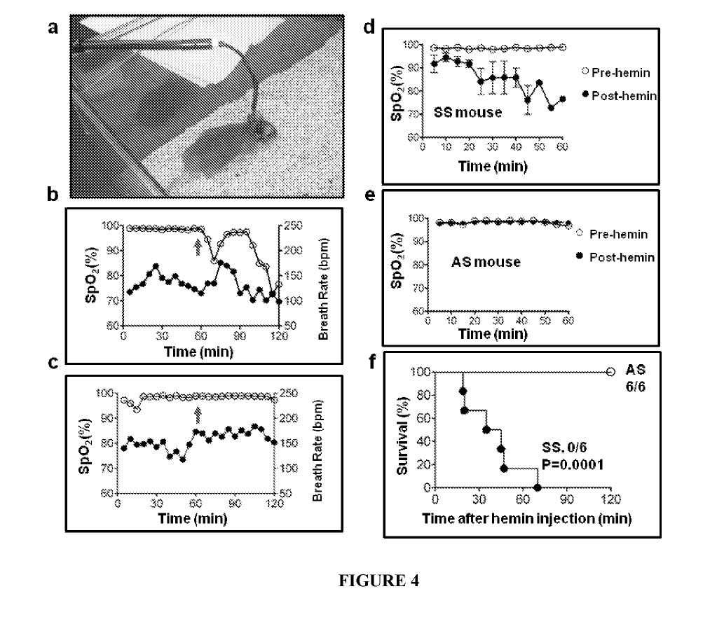

Additionally, a non-invasive approach to correlate oxygen saturation levels to lung pathology due to hemin injection was applied. This approach, which involves attaching a non-invasive neck collar clip sensor to detect signals from the carotid arteries of awake mice (FIG. 4a), has been validated in previous studies to correlate oxygen saturation levels with lung pathology in influenza infections (Sidwell et al. Utilization of pulse oximetry for the study of the inhibitory effects of antiviral agents on influenza virus in mice. Antimicrob Agents Chemother. 1992; 36(2):473-476; Verhoeven D, Teijaro J R, Farber D L. Pulse-oximetry accurately predicts lung pathology and the immune response during influenza infection. Virology. 2009; 390(2):151-156). No difference in baseline oxygen saturation (SpO₂) in SS and control mice was found (FIGS. 4b and 4d; 0-60 minutes). However, intravenous injection of hemin at 70 μmoles/kg caused a sharp drop in SpO₂ concurrent with an increasing breath rate in SS mice (FIG. 4a; 60-120 minutes), but not in AS mice (FIG. 4c; 60-120 minutes). The SpO₂ values returned to physiological levels in SS mice, remained steady for 10-30 minutes, and then began a steady decline until the mice succumbed; this pattern, including the inevitable death, was the same in all SS mice studied to date (FIG. 4d). On the contrary, there is no significant change in SpO₂ or breath rate in AS mice injected with the same dose of hemin (FIG. 4e) Thus, acute elevation of free heme induces hypoxemia and respiratory distress in SS mice, but not in control littermates that have sickle cell trait.

### Example 2

### HO-1 Levels are Elevated in SCD Mice and Patients, and Decrease with Age

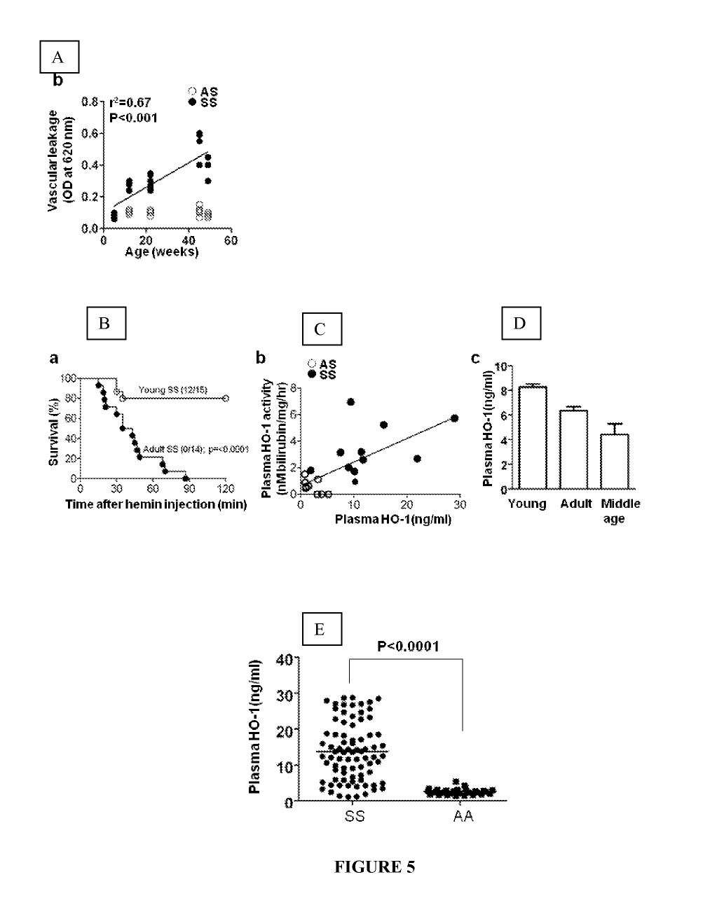

The relationship between plasma levels of heme oxygenase-1, subject age, and survival rates following free heme-induced ACS was analyzed. To determine vascular leakage levels in SS and AS mice over varying ages, mice were injected via the tail vein with 200 μl of 1% Evans blue dye, and allowed free access to food and water for 45 minutes. The dye was cleared from the circulation by perfusion, lungs were harvested and incubated in formamide for 3 days to extract extravasated dye, and vascular leakage was determined through the optical density of the extracted dye at 620 nm. The data indicate an age-dependent increase in vascular leakage in the SS mouse lungs, and no increase in vascular leakage in AS mice from the initial, basal rate (FIG. 5a). Additionally, survival rates of young (5-7 weeks) and adult (13-27 weeks) SS mice that underwent free heme-induced ACS through plasma hemin injection at 70 μmoles/kg were analyzed. Indeed, free heme-induced ACS was associated with 80% survival in young SS mice (5-7 weeks old), contrary to the 100% mortality in adult SS mice aged 13-27 weeks (FIG. 5b).

Plasma HO-1 levels in AS versus SS mice, as well as levels in SS mice of varying ages was also determined by measuring plasma HO-1 concentrations against plasma HO-1 activity in samples taken from said mice. Results showed that the concentration and activity of the enzyme was nearly 6-fold higher in the SS mice (FIG. 5c), and that average plasma HO-1 was modestly higher in young SS mice aged 5-7 weeks than in adult mice, and this value decreased further in middle-aged mice (FIG. 5d). Additionally, plasma HO-1 levels in SCD patients were studied. The mean concentration in a cohort of SCD patients at steady-state, in the absence of acute illness, was 13.7 ng/ml±8.2. This value was 5-fold higher than the mean (2.6 ng/ml±0.8) in an age- and ethnic-matched control group of blood donors (FIG. 5e), as well as the baseline mean (2.3 ng/ml±0.3) reported for volunteers in a recent clinical study of pharmacological activation of HO-1 (Bharucha et al. First-in-human study demonstrating pharmacological activation of heme oxygenase-1 in humans. Clin Pharmacol Ther. 2010; 87(2):187-190). However, there was a 25-fold variation in plasma HO-1 level among the SCD group (range 1.1 ng/ml to 28.7 ng/ml). This variation is likely related to polymorphisms in the HO-1 gene, such as a (GT) microsatellite (Yamada et al. Microsatellite polymorphism in the heme oxygenase-1 gene promoter is associated with susceptibility to emphysema. Am J Hum Genet. 2000; 66(1):187-195; Chen et al. Microsatellite polymorphism in promoter of heme oxygenase-1 gene is associated with susceptibility to coronary artery disease in type 2 diabetic patients. Hum Genet. 2002; 111(1):1-8; Hirai et al. Microsatellite polymorphism in heme oxygenase-1 gene promoter is associated with susceptibility to oxidant-induced apoptosis in lymphoblastoid cell lines. Blood. 2003; 102(5):1619-1621), which is associated with plasma HO-1 level in ARDS patients, and other critically ill patients (Sheu et al. Heme oxygenase-1 microsatellite polymorphism and haplotypes are associated with the development of acute respiratory distress syndrome. Intensive Care Med. 2009; 35(8):1343-1351; Saukkonen et al. Heme oxygenase-1 polymorphisms and plasma concentrations in the critically ill patients. Shock. Apr. 6, 2010 pubmed). Thus, while HO-1 is upregulated in subjects with SCD (likely to scavenge extracellular hemin resulting from increased hemolysis), HO-1 levels decrease with age. These results may also explain why ACS is markedly less severe in children with SCD.

### Example 3

### TLR-4 Mutant Mice Survive Hemin Injection, TLR-4 and NF-κB are Involved in the Free Heme-Induced Inflammation of ACS

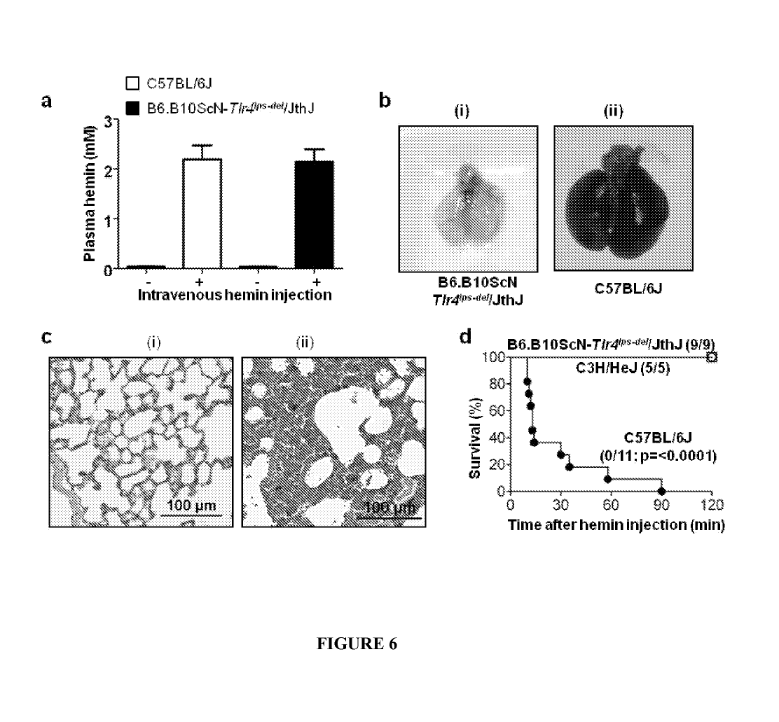

The role of TLR-4 in response to an acute increase in free heme was investigated. Wild type C57BL/6J mice and TLR-4 mutant mice B6.B10ScN-Tlr4^{lps-del} and C3H/HeJ (Poltorak et al. Defective LPS signaling in C3H/HeJ and C57BL/10ScCr mice: mutations in Tlr4 gene. Science. 1998; 282(5396):2085-2088.; Qureshi et al. Endotoxin-tolerant mice have mutations in Toll-like receptor 4 (Tlr4). The J Expt. Med. 1999; 189(4):615-625) were injected intravenously with 210 μmoles/kg hemin, a normally lethal dose. This injection increased free heme concentrations by a magnitude of 50-fold in both wild type and TLR-4 mutant mice (FIG. 6a). FIG. 6b (i) shows that the lungs of the TLR4-mutant mice remained normal after the injection with hemin, while the wild-type mouse lungs was overtly hemorrhagic (FIG. 6b (ii). These gross anatomic findings were corroborated by detailed histology, showing virtually clear alveolar septal networks in the TLR4-mutant mouse (FIG. 6c (i), and blood-filled alveolar in wild-type mice (FIG. 6c (ii). Wild type mice showed 100% mortality rate in response to the hemin injection after 90 minutes; however, both types of TLR-4 mutant mice exhibited 0% mortality in response to the injection (FIG. 6d). This result strongly implicates TLR-4 in the lung pathology, respiratory distress and mortality associated with ACS in response to an acute increase in free heme.

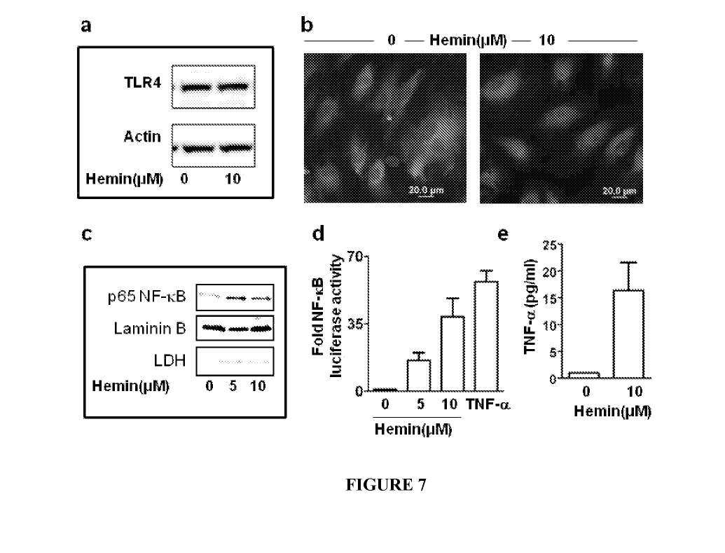

Additionally, the role of NF-κB in the inflammatory response to free heme that occurs in ACS was investigated. Endothelial TLR4, and not neutrophil TLR4, mediates the early stage of the acute sequestration of neutrophils into the lungs in response to systemic LPS (Andonegui et al. Endothelium-derived Toll-like receptor-4 is the key molecule in LPS-induced neutrophil sequestration into lungs. J Clin Invest. 2003; 111(7):1011-1020; Andonegui et al. Mice that exclusively express TLR4 on endothelial cells can efficiently clear a lethal systemic Gram-negative bacterial infection. J Clin Invest. 2009; 119(7): 1921-1930). Thus, pulmonary microvascular endothelial cells (PMVECs) may play a role in the inflammatory response to free heme. We confirmed that PMVECs express TLR4 via western blot analysis (FIG. 7a), and found that hemin dose-dependently activates NF-κB as determined by multiple complementary in vitro assays. These include nuclear translocation of p65 NF-κB in PMVECs as determined by immunofluorescence staining and western blot of nuclear extract (FIGS. 7b and 7c, respectively), luciferase reporter gene activity driven by a promoter harboring NF-κB cis elements in PMVECs (FIG. 7d), and TNF-α production (FIG. 7e). These results in PMVECs are consistent with published studies of the activation of TLR4 by heme in peritoneal macrophages (Figueiredo et al. Characterization of heme as activator of Toll-like receptor 4. J Biol Chem. 2007; 282(28):20221-20229), and further indicate that NF-κB plays a role in the free heme-induced inflammation of ACS.

### Example 4

### Impaired Heme Scavenging is Associated with Development of ALI in SS Mice

Heme is present in many molecules and macromolecular complexes in the bloodstream

including methemalbumin, methemoglobin, cell-free hemoglobin, heme/hemopexin, hemoglobin/haptoglobin, heme/α1-microglobulin, heme/lipid complexes, as well as in an unbound form free from protein. Each of these forms contributes to the concentration of heme measured in unfractionated plasma and reported variously as heme and free heme by others. Total plasma heme (TPH) refers to heme measured in unfractionated plasma. The concentration of TPH was markedly higher in homozygous SCD transgenic mice (SS) than in littermates with sickle cell trait (AS), and in AA transgenic mice expressing normal human hemoglobin (FIG. 8a). The concentration of unbound freely circulating heme has not previously been determined in SCD, in patients or in murine models. Plasma samples were separated by centrifugation through 3000 dalton size-exclusion filters and the heme content in the resulting protein-free fraction, referred hereto as protein-free plasma heme (PFPH) determined. PFPH was virtually undetectable in AA mice but was present at higher levels in SS as well as in AS mice (FIG. 8b).

PFPH has not previously been associated with any specific pathology in SCD. SS mice were intravenously injected with a dose-range (17.5-70 μmoles/kg) of freshly prepared Fe(III)PPIX (heme), which raised TPH and PFPH in a dose-dependent manner (FIG. 8c). While the concentration of PFPH increased markedly in all SS mice given 70 μmoles/kg heme, AS and AA mice effectively scavenged the same dose of heme with no appreciable increase in PFPH (FIG. 8d). Because severe acute hemolysis is associated with lethal forms of ACS, lung function was assessed to determine whether excess PFPH is involved in this process.

There was no basal difference in oxygen saturation (SpO2) and breath rate in SS and AS mice, however a sustained and significant decline in SpO2 occurred in SS mice fifteen minutes after heme administration, and it worsened with time (FIG. 8e), concurrent with reductions in breath rate (FIG. 8f). None of these abnormal lung changes were observed in AS littermates. Heme can convert oxyhemoglobin to methemoglobin. The latter interferes with SpO2 measurements; however, no irregular recordings of SpO2 were found in these initial experiments. Heme caused lethality in SS mice in a dose-dependent manner (FIG. 8g) but had no impact on survival in both AS and AA mice (FIG. 8h) in agreement with lung physiology data.

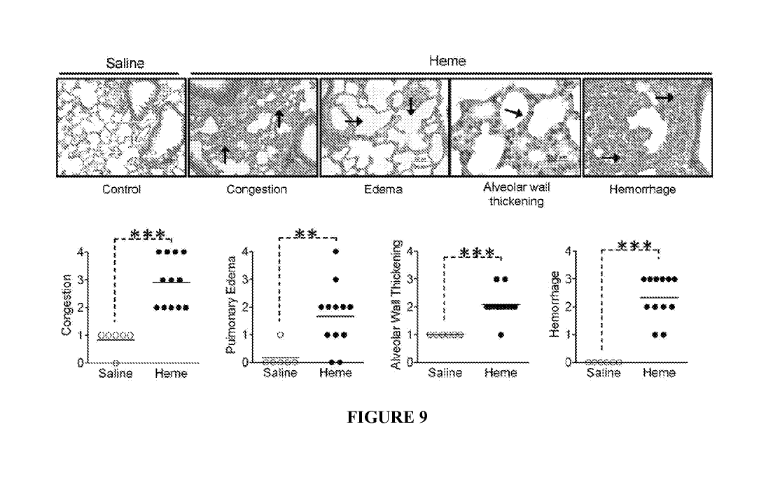

Post mortem analysis of lungs of SS mice that succumbed to heme showed vascular congestion, alveolar edema, alveolar wall thickening and hemorrhage, findings consistent with the reductions in SpO2 and breath rate recorded in the same animals (FIG. 9). Heme scavenged from the circulation is delivered and degraded primarily in the liver. Excess heme accumulation in this organ causes inflammation, apoptosis and necrosis, and implicated in lethality during sepsis. At the median time of death, serum ALT was normal, excluding liver involvement in the sudden lethality of SS mice treated with heme in this study, and this interpretation was confirmed by normal liver histology in post-mortem tissues. The deleterious effects of heme were not unique to the Townes SS mice, as similar findings were made in the Berkeley SCD mouse model. Collectively, these results provide strong evidence that heme scavenging in transgenic SCD mice is severely impaired and it is associated with a lethal form of ALI.

### Example 5

### Hemopexin Deficiency Promotes ALI Development in SS Mice

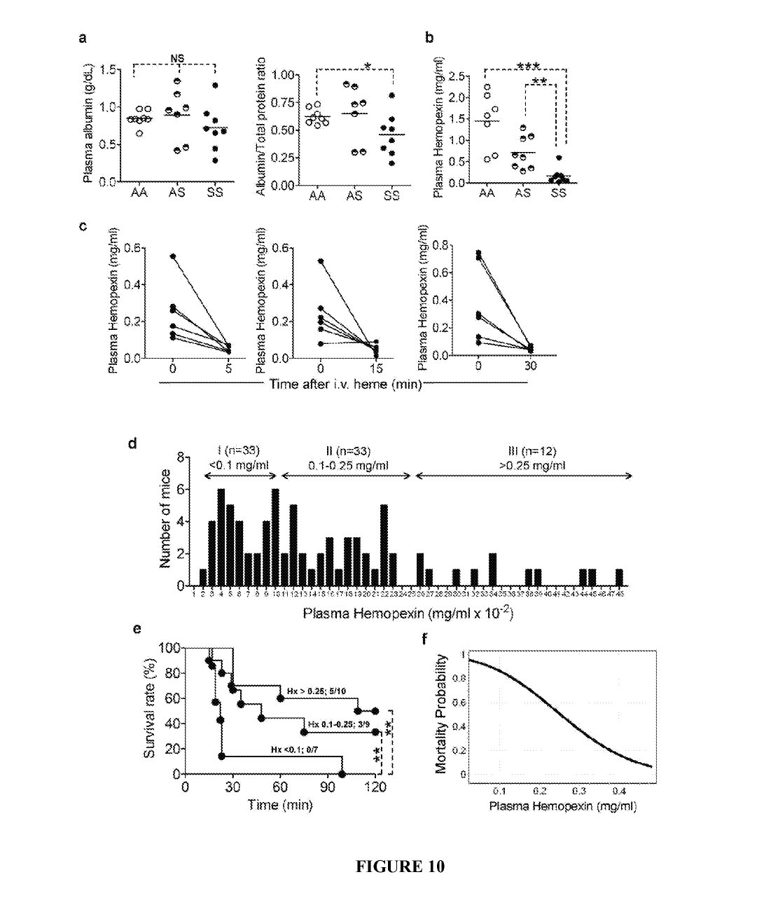

Albumin and hemopexin are the two major plasma scavengers of heme. Formation of methemalbumin reduces the oxidative effects of heme in SCD. The albumin concentration in SS mice was marginally reduced suggesting that this was likely not the major heme scavenging impairment in SCD (FIG. 10a). There is a paucity of studies of plasma hemopexin in SCD. Investigations of small cohorts of patients found that the majority had baseline values less than 0.2 mg/ml. In agreement with those findings, the mean value of hemopexin in the cohort of SS patients was 0.2±0.02 mg/ml (n=12), which was 5-fold lower than the mean value (1.0±0.2 mg/ml, n=12) for age and ethnic matched blood donors with Hb AA. In common with the human disease, the concentration of plasma hemopexin in SS mice was severely low (FIG. 10b). Injections of heme depleted these low reserves further, to virtually undetectable levels (FIG. 10c), and interestingly, by a timeframe that paralleled the onset of SpO2 reductions in our earlier experiments (FIG. 8e).

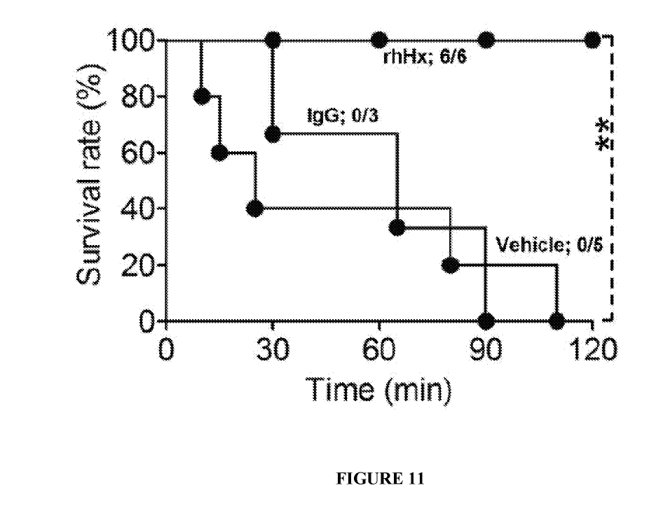

A large number of SCD mice of both the Townes (SS, n=38) and Berkeley (sickle, n=40) models were screened to further investigate the relationship between hemopexin and ALI. Both models had comparably low hemopexin levels with wide variations that allowed categorization of the mice into three groups based on arbitrary cutoff values (FIG. 10d). Acute elevation of plasma heme revealed a strong association between the baseline plasma hemopexin concentration and lethality in a cohort of 26 SS mice. The group of mice with the highest baseline hemopexin level had a 50% survival, while none of the mice in the group with the lowest values (<0.1 mg/ml) survived (FIG. 10e). This data allowed us to extrapolate the probability of survival as a function of plasma hemopexin concentration (FIG. 11). While recognizing that multiple factors influence the development of heme induced ALI, two unique correlative data sets in this study, involving AS mice in FIG. 1h, and SS mice with variable plasma hemopexin level (FIG. 10e), strongly argued for hemopexin playing a dominant role in this process. The plasma concentration of hemopexin determined for AS mice in this study was comparable to those reported in a small cohort of individuals with Hb AS (sickle cell trait) (FIG. 10b).

To determine whether these levels would protect SS mice from heme induced ALI, a cohort of SS mice were intravenously injected with 1 mg of recombinant human hemopexin (to raise their plasma levels to comparable AS values) (n=6), while control groups were given human IgG (n=3) or vehicle (n=5), immediately before elevating plasma heme. The hemopexin-treated mice were all protected while the control mice succumbed to heme (FIG. 11). Thus, acute prophylaxis using hemopexin may be an attractive therapeutic strategy to block the deleterious effects of PFPH during severe episodes of acute hemolysis in SCD.

### Example 6

### Heme Induced ALI Utilizes a Functional TLR4

The lethal ALI phenotype defined here was associated with elevations in PFPH and not

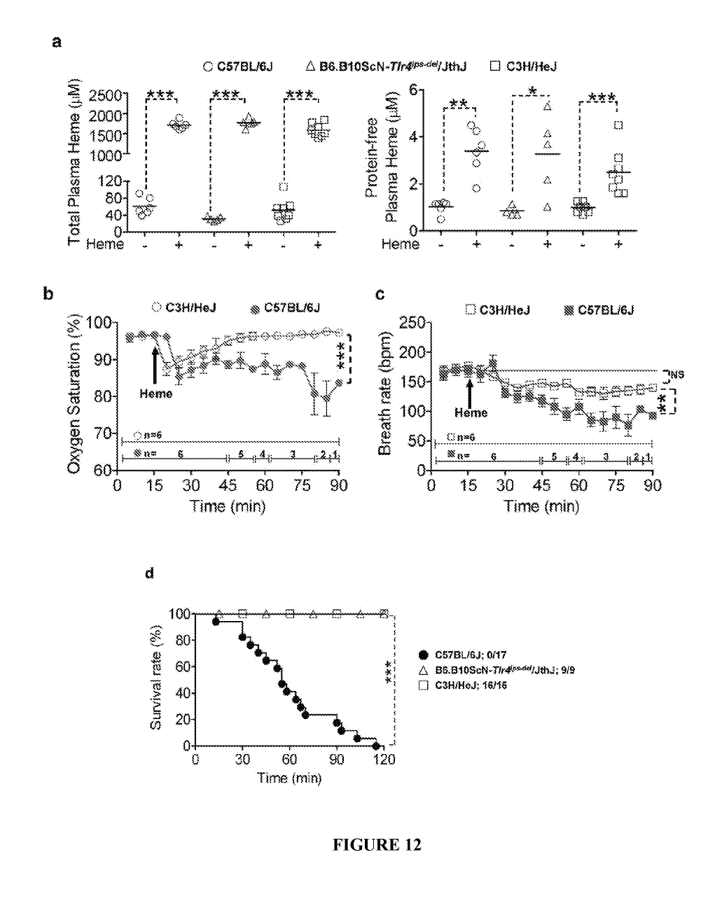

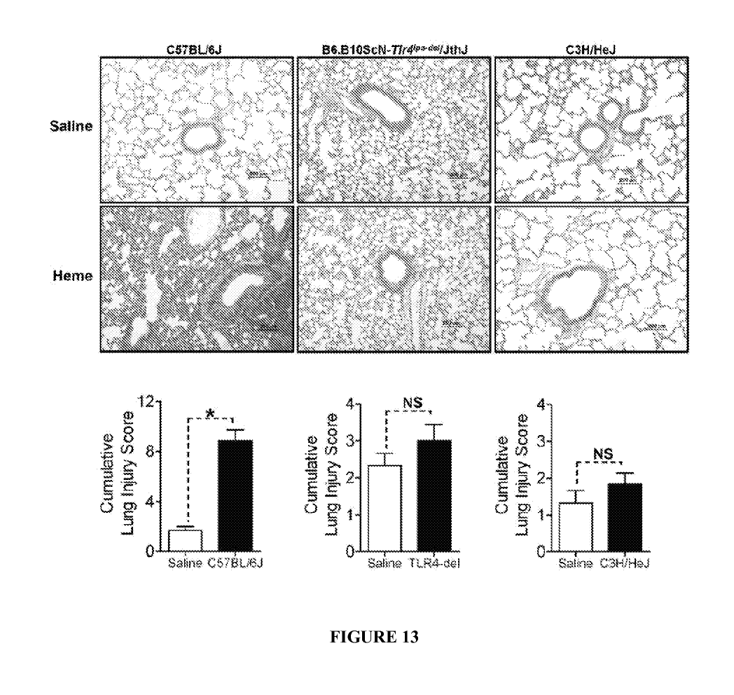

TPH per se, therefore, the involvement of a receptor mediated mechanism was suspected. Several heme receptors have been characterized, including TLR433, which is uniquely expressed by multiple cells in the circulation and therefore accessible to PFPH. Studies were performed in wild-type C57BL/6J mice, and mutant strains, B6.B10ScN-Tlr4lps-del/JthJ and C3H/HeJ, harboring a deletion and a point mutation respectively, in the murine TLR4 gene. Significant elevation of PFPH in these mice required a large dose of heme (210 μmoles/kg) (FIG. 12a), and as expected this amount of heme interfered, transiently with SpO2 measurements characterized by an immediate and dramatic decline. Despite this potential interference, SpO2 values of C57BL/6J mice were consistently lower compared to C3H/HeJ suggesting a biological difference existed in the response of wild-type and TLR4 mutant mice to heme (FIG. 12b). This interpretation was supported by a return of SpO2 values to normal in C3H/HeJ but not in the wild-type C57BL/6J mice, indeed, SpO2 deteriorated over time in the latter reminiscent of the decline in SS mice (FIG. 12b). Heme had no significant overall impact on breath rate in C3H/HeJ but markedly reduced breath rate in C57BL/6J (FIG. 12c). These unique pulmonary responses were consistent with survival (FIG. 12d) and lung histology (FIG. 13). A previous extensive in vitro study showed that other porphyrins do not activate TLR and in agreement with that study, two heme analogues, sodium protoporphyrin IX (Na-PPIX, n=6) and Iron mesoporphyrin (Fe(III)MPP-IX, n=7), injected at the LD100 of heme, had no impact on survival in C57BL/6J. These results provide the first evidence, to the best of our knowledge that the specific interaction between heme and TLR4 can be pathological.

### Example 7

### Resatorvid Prevents the Development of Heme Induced ALI

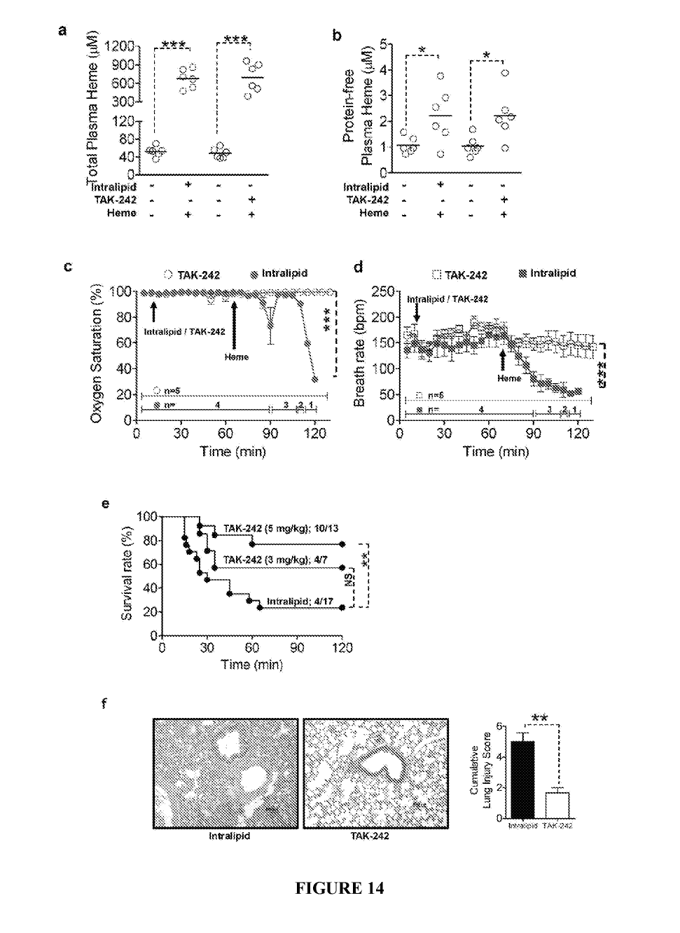

TAK-242 (resatorvid) is a small molecule that binds to Cys747 of the intracellular domain of TLR4 and inhibits downstream signaling. It inhibits the production of lipopolysaccharide (LPS)-induced inflammatory mediators, and it improves survival in LPS-induced sepsis. TAK-242 and an intralipid vehicle did not alter scavenging of heme in SS mice (FIG. 14a, b). One hour prior to heme administration, a cohort of SS mice were intravenously administered 5 mg/kg of TAK-242, or intralipid. The relatively small dose of heme did not produce any irregularities in SpO2 measurements (FIG. 14c). Heme had no impact on SpO2 in the majority of TAK-242 treated SS mice (5/6), however it caused an initially subtle yet sustained decrease that deteriorated over time in the intralipid treated mice (FIG. 14c). Breath rate remained essentially the same in TAK-242-treated SS mice, but it reduced over-time in intralipid treated controls (FIG. 14d). TAK-242 protected 83% of the SS mice from heme induced lethality, compared to 20% in the group given intralipid. These survival data were similar to those obtained in subsequent experiments focused on heme induced lethality (TAK-242; 5/7 (71%) protection, intralipid; 3/12 (20%) protection). The combined data is shown in FIG. 14e, and is supported by histological analysis (FIG. 14f). These results corroborate findings in C57BL/6J and TLR4 mutants, and provide strong preclinical data to support studying TLR4 inhibition as a novel therapeutic strategy in SCD.

1. A method of treating or preventing organ inflammation comprising administering heme scavenger to a subject in need thereof.

2. The method of claim 1, wherein the organ inflammation is pulmonary inflammation.

3. The method of claim 1, wherein the heme scavenger is hemopexin.

4. The method of claim 1, wherein the subject is diagnosed with higher than normal levels of protein-free plasma heme.

5. The method of claim 1, wherein the subject is diagnosed with a hemolytic disorder.

6. The method of claim 5, wherein the hemolytic disorder is sickle cell disease, autoimmune hemolytic anemia, malaria or paroxysmal nocturnal hemoglobinuria.

7. The method of claim 1, wherein the subject is diagnosed with acute lung injury or acute chest syndrome.

8. The method of claim 3, wherein the hemopexin is recombinant human hemopexin.

9. A method of treating or preventing acute pulmonary inflammation comprising administering a TLR4 antagonist to a subject in need thereof.

10. The method of claim 9, wherein the subject is diagnosed with higher than normal levels of protein-free plasma heme.

11. The method of claim 9, wherein the subject is diagnosed with a hemolytic disorder.

12. The method of claim 11, wherein the hemolytic disorder is sickle cell disease, autoimmune hemolytic anemia, or paroxysmal nocturnal hemoglobinuria.

14. The method of claim 9, wherein the subject is diagnosed with acute lung injury or acute chest syndrome.

15. The method of claim 9, wherein the TLR4 antagonist is eritoran or resatorvid.

16. A method of identifying a heightened risk of developing acute pulmonary inflammation comprising measuring plasma hemopexin in a subject and correlating a higher than normal level of hemopexin to a heightened likelihood of developing pulmonary inflammation.

17. The method of claim 16 further comprising the step of reporting the plasma hemopexin level or a heightened risk of developing acute pulmonary inflammation to the subject or a medical professional.
